(19) United States
(12) Patent Application Publication
(10) Pub. No.: US 2025/0019725 A1
(43) Pub. Date: Jan. 16, 2025

(54) METHOD FOR PRODUCING SYNGAS FERMENTATION PRODUCTS USING HIGHLY ACTIVE MICROORGANISMS

(71) Applicant: GWANGJU INSTITUTE OF SCIENCE AND TECHNOLOGY, Gwangju (KR)

(72) Inventors: In Seop CHANG, Gwangju (KR); Mun Gyu LEE, Gwangju (KR); Ji Yeon KIM, Gwangju (KR); So Young OH, Gwangju (KR); Nulee JANG, Gwangju (KR)

(21) Appl. No.: 18/767,374

(22) Filed: Jul. 9, 2024

(30) Foreign Application Priority Data
Jul. 11, 2023 (KR) 10-2023-0089691

(51) Int. Cl. C12P 5/02; C12M 1/00; C12M 1/34; C12N 1/20

(52) U.S. Cl. CPC C12P 5/02; C12M 23/40; C12M 23/58; C12M 29/08; C12M 41/26; C12M 41/30; C12N 1/20

(57) ABSTRACT

An embodiment relates to a method for producing syngas fermentation products using highly active microorganisms, and in particular, to a method for producing syngas fermentation products using highly active microorganisms capable of maximizing production efficiency of fermentation products by obtaining microorganisms having a highly active cell concentration through a biomass boosting step in a bioreactor and using these in a fermentation process in a separate bioreactor.

### CROSS-REFERENCE TO RELATED APPLICATIONS

This application claims the priority of Korean Patent Application No. 10-2023-0089691 filed on Jul. 11, 2023, in the Korean Intellectual Property Office, the disclosure of which is incorporated herein by reference.

### Technical Field

The present disclosure relates to a method for producing syngas fermentation products using highly active microorganisms.

### Background Art

In the ranking of domestic industrial production, a petroleum-based energy/chemical industry occupies a position of domestic representative key industry, however, due to factors such as recent price instability and supply and demand of petroleum with repeated stable and unstable trends, demands for securing fundamental technology for discovering and utilizing replaceable raw materials that are stable and inexpensive are rapidly increasing. Currently, C1 gas including methane (CH₄), carbon dioxide (CO₂) and carbon monoxide (CO) formed with one carbon atom, which has huge amounts of reserves but is only utilized through simple combustion, is formed with carbon and oxygen like petroleum in terms of molecular structure, and may be converted into various transportation fuels and chemical materials. Therefore, technology for converting to transportation fuels and chemical materials using C1 gas has an enormous ripple effect upon successful development, and research and development on related technologies is competitively ongoing recently.

However, existing C1 gas conversion processes lack process economic feasibility compared to existing petroleum-based chemical processes due to low yield and high operating costs. It is known that, since an existing chemical conversion process of C1 gas based on indirect conversion is performed under a harsh condition of high temperature and high pressure, local overheating occurs without proper discharge of generated heat, and serious problems such as catalyst deterioration and reactor cracking may be caused. In addition, since C1 gas such as CO or CH₄ is highly reactive to oxidizing reagents, a precise reaction control at the molecular level is essential in order to prevent a decrease in the yield of target products caused by by-product generation, and continuous studies thereon are in progress.

Recently, clues on high-performance biocatalyst and chemical catalyst designs capable of economically converting C1 gas have been provided as basic research results on molecular biology or C1 chemistry have accumulated. In particular, a bioconversion process of C1 gas is considered as high value-added technology since useful substances may be produced through a C1 gas direct conversion path, which is theoretically the simplest reaction path.

In addition, unlike common chemical processes, the reaction proceeds under a mild condition of low temperature and low pressure, and, for microbial species used herein, it is reported that a wide variety of microbial species such as methanogenic bacteria, methanotrophic bacteria and acetogenic bacteria may be used. Unlike microorganisms such as Saccharomyces cerevisiae(S. cerevisiae) or Escherichia coli (E. coli) that grow or ferment using sugar as a substrate, these specially use C1 gas or by-product gas emitted during combustion such as carbon monoxide (CO), carbon dioxide (CO₂) and methane (CH₄) to produce various raw materials such as industrially valuable bio-methanol (CH₃OH), bio-hydrogen (H₂), propanol (C₃H₆OH), acetic acid (CH₃COOH), succinic acid ((CH₂)₂(CO₂H)₂), maleic acid (C₄H₄O₄) and formic acid (HCOOH).

However, a Cl gas conversion microbial process has very low C1 gas solubility (carbon monoxide: 20 mg/L to 25 mg/L, CH₄: 15 mg/L to 20 mg/L), and, even when there is an excellent bioconversion strain or catalyst, has a fundamental limit in improving productivity in the absence of process technology capable of improving mass transfer of C1 gas, which is a fundamental problem. In this regard, various high-concentration cell culture methods such as cell recycling and membrane reactor have been proposed, and various studies to overcome the problem of mass transfer are in progress.

### [Prior Art Documents]

### [Patent Documents]

(Patent Document 1) KR 10-2014-0063773 A

### DISCLOSURE

### Technical Problem

The present disclosure is directed to providing a method for producing syngas fermentation products using highly active microorganisms.

The present disclosure is also directed to providing a method for producing syngas fermentation products using highly active microorganisms capable of maximizing production efficiency of fermentation products by obtaining microorganisms having a highly active cell concentration through a biomass boosting step in a bioreactor and using these in a fermentation process in a separate bioreactor.

The present disclosure is also directed to providing a system for producing syngas fermentation products using highly active microorganisms.

### Technical Solution

In view of the above, a method for producing syngas fermentation products using highly active microorganisms according to one embodiment of the present disclosure includes: a biomass boosting step of obtaining highly active microorganisms by injecting a gaseous substrate including CO into a first bioreactor; a step of supplying the obtained highly active microorganisms to a second bioreactor; and a product producing step of injecting a gaseous substrate into the second bioreactor, and producing hydrocarbon products by a fermenting action of the highly active microorganisms in the second bioreactor, wherein the first bioreactor includes a first culture medium, and the second bioreactor includes a second culture medium.

A pH condition inside the first and second bioreactors is from 6.8 to 7.2.

A first culture medium addition rate (dilution rate) in the first bioreactor is from 0.08 h⁻¹ to 0.15 h⁻¹.

A second culture medium addition rate (dilution rate) in the second bioreactor is from 0.01 h⁻¹ to 0.02 h⁻¹.

A gaseous substrate injection rate (vvm) into the first bioreactor is from 0.06 vvm to 0.86 vvm.

A gaseous substrate injection rate (vvm) into the second bioreactor is from 0.086 vvm to 0.1 vvm.

The first and second culture media may include a compound selected from the group consisting of NaCl, MgSO₄·7H₂O, CaCl₂·2H₂O, NH₄Cl and mixtures thereof.

The microorganism is selected from the group consisting of genus Moorella, Clostridium, Ruminococcus, Acetobacterium, Eubacterium, Butyribacterium, Oxobacter, Methanosarcina, Methanosarcina and Desulfotomaculum.

The hydrocarbon product is selected from the group consisting of acetate, butyrate, ethanol, propanol, butanol, 2,3-butanediol, propionate, caproate, propylene, butadiene, isobutylene, ethylene and mixtures thereof.

A hydrocarbon product according to another embodiment of the present disclosure is produced using the method for producing syngas fermentation products.

A system for producing syngas fermentation products using highly active microorganisms according to another embodiment of the present disclosure includes: a first bioreactor; a second bioreactor; a gas cylinder capable of injecting a gaseous substrate including CO into the first bioreactor and the second bioreactor; and a feed tank connected to each of the first bioreactor and the second bioreactor and capable of controlling a medium addition rate (dilution rate), wherein highly active microorganisms obtained in the first bioreactor are supplied to the second bioreactor connected to the first bioreactor, and the gaseous substrate is injected and fermented to produce hydrocarbon products.

To the second bioreactor, a hollow fiber membrane may be additionally connected to concentrate the microorganisms.

The first and second bioreactors are capable of independently controlling one or more variables selected from the group consisting of the medium addition rate, the gaseous substrate injection rate, a pH inside the reactors and combinations thereof.

A base tank is connected to the first and second bioreactors, and by the base tank, a pH inside the first and second bioreactors is maintained at 6.8 to 7.2.

The term “gaseous substrate” in the present specification is a gaseous substance including CO, and may include all of a waste gas itself including CO and CO₂, or CO, CO₂, H₂ or a mixture gas thereof purified and separated from the waste gas.

Meanwhile, the “waste gas” may be a waste gas obtained as a by-product of an industrial process or from other supply sources such as automobile exhaust fumes or biomass gasification. Specifically, the “industrial process” may be selected from the group consisting of ferrous metal product manufacturing such as steel manufacturing, non-ferrous metal product manufacturing, petroleum refining, coal gasification, electric power production, carbon black production, ammonia production, methanol production and coke manufacturing.

### Advantageous Effects

The present disclosure relates to a method for producing syngas fermentation products using highly active microorganisms, and the method is capable of maximizing production efficiency of fermentation products by obtaining microorganisms having a highly active cell concentration through a biomass boosting step in a bioreactor and using these in a fermentation process in a separate bioreactor.

### BRIEF DESCRIPTION OF DRAWINGS

FIG. 1 shows results of identifying production efficiency of hydrocarbon products using a method for producing fermentation products according to one embodiment of the present disclosure.

FIG. 2 shows results of identifying microbial activity obtained from performing a biomass boosting step in a separate reactor in a method for producing fermentation products according to one embodiment of the present disclosure.

FIG. 3 illustrates a schematic diagram of a system for producing syngas fermentation products using highly active microorganisms according to one embodiment of the present disclosure.

### Best Mode

Hereinafter, embodiments of the present disclosure will be described in detail so that those skilled in the art may readily implement the present disclosure. However, the present disclosure may be embodied in various different forms, and is not limited to the embodiments described herein.

Biological conversion of syngas, that is, syngas fermentation, is receiving attention as a promising alternative to existing processes by producing fuels and chemicals in an environmental-friendly way, and has advantages of lower raw material supply costs compared to chemical conversion methods, and relatively low energy consumption required for process operation.

Nevertheless, a biological conversion pathway of syngas using microorganisms still has unresolved problems in that there are problems such as poor gas-liquid mass transfer (GL-MT) and low production yield.

Accordingly, various studies have been conducted to resolve the problem of poor gas-liquid mass transfer (GL-MT) and increase product concentration in syngas fermentation, and the present applicant has developed a process system capable of simultaneously increasing a high microbial cell concentration and a production concentration of fermentation products by integrating a bubble column reactor (BCR) capable of exhibiting a high k_{L}a (volumetric mass transfer coefficient) and a continuous cell recycled reactor (CCR) capable of achieving a high microbial cell concentration.

Meanwhile, since a continuous process of performing a biomass boosting step that increases a cell concentration of highly active microorganisms and a fermentation product producing step is performed inside one bubble column reactor in the system, the concentration of old cells having low activity increases in the reactor as the process progresses, and as a result, production efficiency of fermentation products may be reduced.

In other words, as the concentration of microorganisms increases in the reaction process, more fermentation products may be produced theoretically since the concentration of microorganisms capable of producing fermentation products increases, however, the production yield does not increase in proportion to the increase in the concentration of microorganisms, and this is due to the fact that, even with the same cell concentration, the production yield of products varies depending on the activity of microorganisms.

In view of the above, the present applicant has designed the process so that the biomass boosting step and the fermentation product producing step are each performed in separate reactors. Through this, a highly active cell concentration in the reactor where the fermentation step is performed is secured by continuously supplying highly active microorganisms to a reactor where the fermentation step is performed, and through this, a novel process capable of improving production efficiency of fermentation products has been developed.

A method for producing syngas fermentation products using highly active microorganism according to one embodiment of the present disclosure includes: a biomass boosting step of obtaining highly active microorganisms by injecting a gaseous substrate including CO into a first bioreactor; a step of supplying the obtained highly active microorganisms to a second bioreactor; and a product producing step of injecting a gaseous substrate into the second bioreactor, and producing hydrocarbon products by a fermenting action of the highly active microorganisms in the second bioreactor, wherein the first bioreactor includes a first culture medium and microorganisms, and the second bioreactor includes a second culture medium.

Specifically, the method for producing syngas fermentation products using highly active microorganisms of the present disclosure includes a biomass boosting step of obtaining highly active microorganisms by injecting a gaseous substrate including CO into a first bioreactor including a first culture medium and microorganisms, and, after going through the biomass boosting step, a step of supplying the highly active microorganisms to a second bioreactor including a second culture medium, and a product producing step of producing hydrocarbon products by injecting and fermenting the gaseous substrate.

Meanwhile, a first culture medium is included in the first bioreactor in the biomass boosting step, and therefore, highly active microorganisms may be obtained by injecting a gaseous substrate including CO and microorganisms.

Inside the second bioreactor, only a second culture medium is initially included, and hydrocarbons may be produced using highly active microorganisms supplied from the first bioreactor.

Meanwhile, as another example, microorganisms as well as a second culture medium may be initially included inside the second bioreactor, and in this case, hydrocarbons may be produced using both the existing microorganisms present inside the second bioreactor and highly active microorganisms supplied from the first bioreactor.

The biomass boosting step means a step of securing a highly active microbial cell concentration capable of maximizing production efficiency of fermentation products by increasing the cell concentration of microorganisms included inside the first bioreactor, and the gaseous substrate and microorganisms may be injected into the first bioreactor including a first culture medium, and the microorganisms may be cultured to secure highly active microorganisms.

More specifically, the “highly active microorganisms” mean microbial cells having a concentration with an OD₆₆₀ value of 4 to 6. In particular, microorganisms having an OD₆₆₀ value that fall within the above-mentioned OD₆₆₀ range value exhibit high activity, and have an advantage of improving production efficiency of fermentation products.

When the concentration is less than the above-mentioned range, there is a problem in that the concentration is not sufficient to produce fermentation products of the present disclosure, and when the concentration is greater than the above-mentioned range, the microbial cells are excessively concentrated, and production yield of fermentation products may decrease.

Meanwhile, the product producing step is a step of performing a fermentation process of a gaseous substrate using highly active microorganisms obtained in the first bioreactor through the biomass boosting step, and producing hydrocarbon products. Specifically, the highly active microorganisms are supplied to the second bioreactor connected to the first bioreactor, and through the fermentation process of a gaseous substrate injected into the second bioreactor, hydrocarbon products may be produced.

Herein, the “highly active microorganisms” supplied to the second bioreactor are microorganisms gone through the biomass boosting step inside the first bioreactor, and mean microbial cells having a concentration with an OD₆₆₀ value of 4 to 6. In other words, during the process of culturing the microorganisms in the first bioreactor, microorganisms having an OD660 value in the range of 4 to 6 may be continuously injected into the second bioreactor, and inside the second bioreactor, the highly active microbial cells may be used in the fermenting step to maximize the production yield of fermentation products.

Specifically, the gaseous substrate including CO may include CO and CO₂, and may include CO and CO₂ in a partial pressure ratio of 3.5:1 to 4.5:1.

Preferably, the product producing step may further include 1) a sludge filtrate injecting step of additionally injecting a sludge filtrate corresponding to 40% to 60% of the medium volume 200 hours to 300 hours after performing the product producing step, and through this, the production yield of hydrocarbon products may increase.

Meanwhile, the sludge filtrate may be injected through a feed tank of the second bioreactor.

Specifically, the sludge filtrate means a liquid component obtained from dehydration when producing sludge cake in a sewage treatment plant.

More preferably, the product producing step may further include 2) a step of adjusting the partial pressure of the gaseous substrate, which injects a gaseous substrate including CO and CO₂ in a partial pressure ratio of 8.5:1 to 9:1 400 hours to 450 hours after performing the product producing step.

Specifically, the gaseous substrate is injected into the second bioreactor through the gas cylinder, and the production yield of hydrocarbon products may be maximized by adjusting the partial pressure ratio of the gaseous substrate.

Meanwhile, the first and second bioreactors are capable of independently controlling one or more variables selected from the group consisting of the medium addition rate, the gaseous substrate injection rate, a pH inside the reactors and combinations thereof, and are able to control to have variables optimized for the biomass boosting step and the product producing step, respectively.

Specifically, the pH condition inside the first and second bioreactors is from 6.8 to 7.2. The first medium addition rate (dilution rate) in the first bioreactor is from 0.08 h⁻¹ to 0.15 h⁻¹.

Specifically, the medium addition rate or dilution rate means a value obtained by dividing the volume of medium supplied per unit time by the volume of incubator, and the growth rate of cells may be controlled by adjusting the medium supply rate or dilution rate in a chemostat that has reached a steady state.

The medium addition rate (dilution rate) in the first bioreactor is larger than the medium addition rate in the second bioreactor, which may increase microbial activity by facilitating growth of microorganisms in the first bioreactor.

More specifically, the first medium addition rate in the first bioreactor is from 0.10 h⁻¹ to 0.15 h⁻¹, and when the rate is more preferably 0.12 h⁻¹, production of highly active microorganisms may be maximized.

The second culture medium addition rate (dilution rate) in the second bioreactor is from 0.01 to h⁻¹ 0.02 h⁻¹.

The medium addition rate (dilution rate) in the second bioreactor may be smaller than the medium addition rate in the first bioreactor, and through this, production efficiency of the hydrocarbon products of the present disclosure may be enhanced by facilitating fermentation of the gaseous substrate in the second bioreactor.

Specifically, the microbial concentration inside the second bioreactor of the present disclosure has an OD₆₆₀ value maintained at a high level (15 to 25), which requires a supply of many nutrients. Accordingly, in order to maintain a constant production concentration of hydrocarbon products from the microorganisms, it is necessary to maintain the dilution rate relatively low.

More specifically, the medium addition rate in the second bioreactor is from 0.015 h⁻¹ to 0.02 h⁻¹, and when the rate is more preferably 0.017 h⁻¹, production efficiency of hydrocarbon products from the microorganisms may be maximized.

The gaseous substrate injection rate (vvm) into the first bioreactor is from 0.06 vvm to 0.86 vvm.

The gaseous substrate injection rate (vvm, volume per volume per minute) represents an injection rate of the gaseous substrate of the present disclosure into the reactor, and the injection rate (vvm) of the gaseous substrate into the first bioreactor is from 0.06 vvm to 0.86 vvm.

When the gaseous substrate is injected in the above-mentioned injection rate range, production of highly active microorganisms targeted in the first bioreactor may be maximized.

More preferably, the injection rate (vvm) of the gaseous substrate into the first bioreactor is gradually increased up to 0.86 vvm. Specifically, after starting the injection at an initial rate of 0.06 vvm, the injection rate is increased by 0.1 vvm per 1 g/L of an increase in the microbial cell concentration in the first bioreactor, and the injection rate may be ultimately increased up to 0.86vvm. In other words, each time the cell concentration increases by 1 g/L, the injection rate may be gradually increased by 0.1 vvm in proportion thereto from the initial injection rate, ultimately reaching 0.86 vvm.

Meanwhile, the gaseous substrate injection rate (vvm) into the second bioreactor is from 0.086 vvm to 0.1 vvm.

When the gaseous substrate is injected in the above-mentioned injection rate range, production efficiency of fermentation products (hydrocarbon products) targeted in the second bioreactor may be maximized.

The first and second culture media may include a compound selected from the group consisting of NaCl, MgSO₄·7H₂O, CaCl₂·2H₂O, NH₄Cl and mixtures thereof.

Preferably, the first and second culture media may include NaCl, MgSO₄·7H₂O, CaCl₂·2H₂O and NH₄Cl.

Meanwhile, components of the culture medium of the present disclosure are defined such that, in addition to the compounds exemplified above, an addition of components that may be additionally included as culture medium components for culturing microorganisms by those skilled in the art is not limited.

Specifically, in the culture medium of the present disclosure, a yeast extract, a vitamin solution, a trace element solution, NaHCO₃, Na₂HPO₄, NaH₂PO₄, L-cysteine·HCl and Resazurin (7-hydroxy-3H-phenoxazin-3-one 10-oxide) may be additionally included, however, the culture medium is not limited thereto.

Meanwhile, components that may be included in the vitamin solution may include biotin, folic acid, pyridoxine·HCl, thiamine HCl, riboflavin, nicotinic acid, pantothenic acid, cyanocobalamine, r-aminobenzoic acid, lipoic acid and the like, and components that may be included in the trace element solution may include nitrilotriacetic acid, FeSO₄·7H₂O, MnCl₂·4H₂O, CoCl₂·6H₂O, ZnCl₂, CaCl₂·6H₂O, CuCl₂·2H₂O, H₃BO₃, Na₂MoO₄, Na₂SeO₃, NiSO4.6H₂O, NaCl and the like. However, the components are not limited to the above-mentioned components, and are not particularly limited as long as they are materials that may be used as components of a vitamin solution and a trace element solution for culturing microorganisms by those skilled in the art.

Preferably, the first bioreactor and the second bioreactor of the present disclosure may include culture media having different compositions or amounts, and the medium included in the first bioreactor may be defined as the first culture medium, and the medium included in the second bioreactor may be defined as the second culture medium.

In other words, in terms that the first bioreactor and the second bioreactor are separate reactors in which the biomass boosting step and the product producing step are respectively performed, optimal compositions and amounts of the culture media for respectively enhancing production of highly active microorganisms and production efficiency of fermentation products may be set.

Preferably, the first culture medium included in the first bioreactor may include NaCl, MgSO₄·7H₂O, CaCl₂·2H₂O, NH₄Cl and the trace element solution in a weight ratio of 1.5:0.3:0.1:1.5:0.03 to 2.5:1.3:0.5:2.5:0.1.

In the above-mentioned weight range, the production of highly active microorganism may be further maximized inside the first bioreactor due to synergistic effects obtained by mixing each component.

Meanwhile, the second culture medium included in the second bioreactor may include NaCl, MgSO₄·7H₂O, CaCl₂·2H₂O, NH₄Cl and the trace element solution in a weight ratio of 5:1.5:0.5:5:0.1 to 10:5:3.5:10:0.5.

In the above-mentioned weight range, production efficiency of hydrocarbon products that are microorganism fermentation products may be maximized inside the second bioreactor due to synergistic effects obtained by mixing each component. In other words, compared to the first culture medium used in the biomass boosting step, a high concentration of culture medium is used to induce effective production of fermentation products.

The microorganism is selected from the group consisting of genus Moorella, Clostridium, Ruminococcus, Acetobacterium, Eubacterium, Butyribacterium, Oxobacter, Methanosarcina, Methanosarcina and Desulfotomaculum.

However, the microorganism is not limited to the microorganism types exemplified above, and is defined to include all microorganism types capable of producing the hydrocarbon products of the present disclosure, which are fermentation products, using the gaseous substrate including CO of the present disclosure.

Specifically, the microorganism of the present disclosure is preferably Eubacterium, and more preferably an Eubacterium limosum KIST612 strain.

Meanwhile, the hydrocarbon product is selected from the group consisting of acetate, butyrate, ethanol, propanol, butanol, 2,3-butanediol, propionate, caproate, propylene, butadiene, isobutylene, ethylene and mixtures thereof.

More specifically, the hydrocarbon product is selected from the group consisting of acetate, butyrate and mixtures thereof.

Meanwhile, a hydrocarbon product according to another embodiment of the present disclosure is produced using the method for producing syngas fermentation products.

In addition, a system for producing syngas fermentation products using highly active microorganisms according to another embodiment of the present disclosure includes: a first bioreactor; a second bioreactor; a gas cylinder capable of injecting a gaseous substrate including

CO into the first bioreactor and the second bioreactor; and a feed tank connected to each of the first bioreactor and the second bioreactor and capable of controlling a medium addition rate (dilution rate), wherein highly active microorganisms obtained in the first bioreactor are supplied to the second bioreactor connected to the first bioreactor, and the gaseous substrate is injected and fermented to produce hydrocarbon products.

Meanwhile, a hollow fiber membrane for concentrating the microorganisms may be additionally connected to the second bioreactor.

The first and second bioreactors are capable of independently controlling one or more variables selected from the group consisting of the medium addition rate, the gaseous substrate injection rate, a pH inside the reactor and combinations thereof.

To the first and second bioreactors, a base tank is connected, and by the base tank, a pH inside the first and second bioreactors is maintained at 6.8 to 7.2.

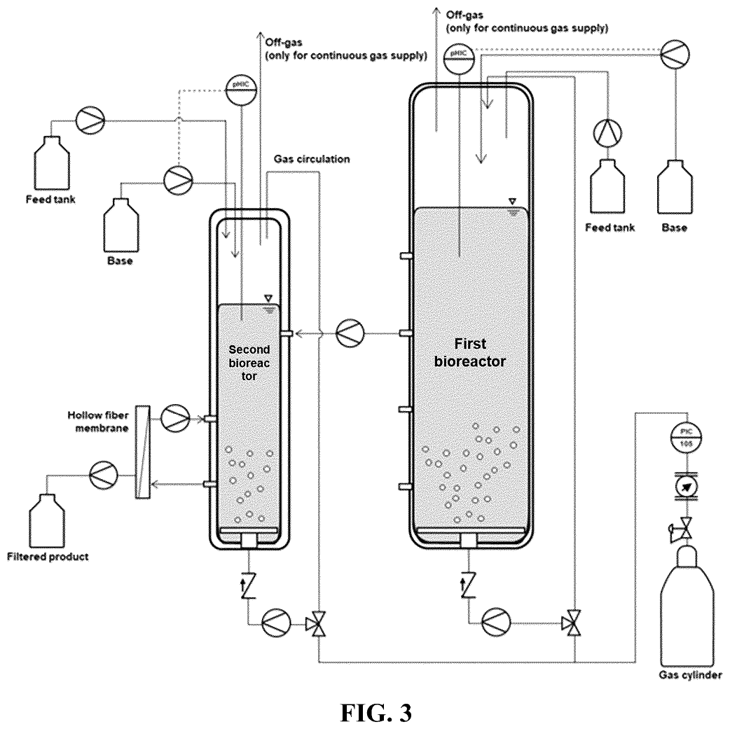

A schematic diagram illustrating the system for producing syngas fermentation products is shown in FIG. 3.

When specifically describing the system of the present disclosure referring to FIG. 3, first and second bioreactors that are independent from each other may be provided. To each of the reactors, a gas cylinder capable of injecting a gaseous substrate is connected, and to each of the reactors, a feed tank capable of controlling a medium addition rate or dilution rate and a base tank capable of controlling a pH inside the reactor may be connected.

Specifically, the first bioreactor is a reactor where a biomass boosting step is performed, and, by injecting the gaseous substrate of the present disclosure including CO into the first bioreactor including microorganisms and a first culture medium, highly active microorganisms are produced. During this process, the pH inside the reactor is maintained at 6.8 to 7.2 by the base tank, and the dilution rate is maintained at 0.10 h⁻¹ to 0.15 h⁻¹ by the feed tank. Meanwhile, the injection rate of the gaseous substrate injected into the first bioreactor may be adjusted by the gas cylinder, and specifically, the injection rate may be increased by 0.1 vvm per 1 g/L of microbial cell concentration in the first bioreactor, and ultimately be adjusted up to 0.86 vvm.

Meanwhile, when highly active microorganisms having a concentration with an OD₆₆₀ value in the range of 4 to 6 are grown by performing a biomass boosting step inside the first bioreactor, these are supplied to the second bioreactor connected to the first bioreactor to perform a product producing step inside the second bioreactor as a continuous process.

More Specifically, the highly active microorganisms include a second culture medium, and when supplied into the second bioreactor into which a gaseous substrate is injected, the highly active microorganisms perform a fermenting step using the substrate and the culture medium to produce hydrocarbon products. Meanwhile, to the second bioreactor, a hollow fiber membrane capable of continuously concentrating the microorganisms is additionally connected, which may further enhance production efficiency of fermentation products inside the second bioreactor.

During the process, the pH inside the second bioreactor is maintained at 6.8 to 7.2 by the base tank, and the dilution rate is maintained at 0.015 h⁻¹ to 0.02 h⁻¹ by the feed tank. Meanwhile, the injection rate (vvm) of the gaseous substrate injected into the second bioreactor may be adjusted to 0.086 vvm to 0.1 vvm by the gas cylinder.

As in the system of the present disclosure, when a biomass boosting step and a product producing step are each performed in independent reactors and highly active microorganisms gone through the biomass boosting step are continuously supplied to the reactor in which the fermenting step is performed, high activity of the microorganisms may be maintained throughout the process, and through this, the production yield of hydrocarbon products that are fermentation products may be enhanced.

Experimental Material Preparation and Method

Preparation of Microbial Strain and Medium

In the experiment, an Eubacterium limosum KIST612 strain was used, and as for culture of the microbial strain and composition of a medium, a known method (Chang et al., 2007; Park et al., 2017) was used.

Meanwhile, specific compositions of the medium used in the present disclosure are shown in the following Tables 1 and 2.

Table 1 shows the composition of the culture medium included in the first bioreactor to be used in the biomass boosting step, and Table 2 shows the composition of the culture medium included in the second bioreactor to be used in the product producing step.

TABLE 1

| CBBM | | | Vitamin solution | | Trace element solution | |
|---|---|---|---|---|---|---|
| | Conc. | | | Conc. | | Conc. |
| Components | (g/L) | | Components | (mg/L) | Components | (g/L) |
| NaCl | 1.8 | | Biotin | 2.0 | Nitrilotriacetic acid | 1.5 |
| MgSO₄•7H₂O | 0.64 | | Folic acid | 2.0 | FeSO₄•7H₂O | 0.1 |
| CaCl₂•2H₂O | 0.3 | | Pyridoxine•HCl | 10.0 | MnCl₂•4H₂O | 0.1 |
| NH₄Cl | 2.0 | | Thiamine•HCl | 5.0 | CoCl₂•6H₂O | 0.17 |
| Yeast extract | 2.0 | | Riboflavin | 5.0 | ZnCl₂ | 0.1 |
| Vitamin solution | 10 | ml | Nicotinic acid | 5.0 | CaCl₂•6H₂O | 0.1 |
| Trace element solution | 20 | ml | Pantothenic acid | 5.0 | CuCl₂•2H₂O | 0.02 |
| Sodium bicarbonate | 2.1 | | Cyanocobalamine | 0.1 | H₃BO₃ | 0.01 |
| Na₂HPO₄ | 1.5883 | | r-aminobenzoic acid | 5.0 | Na₂MoO₄ | 0.01 |
| NaH₂PO₄ | 0.5827 | | Lipoic acid | 5.0 | Na₂SeO₃ | 0.017 |
| L-cysteine•HCl | 0.5 | | | | NiSO₄•6H₂O | 0.026 |
| 0.1% (w/v) resazurin | 200 | μl | | | NaCl | 1.0 |

TABLE 2

| CBBM | | | Vitamin solution | | Trace element solution | |
|---|---|---|---|---|---|---|
| | Conc. | | | Conc. | | Conc. |
| Components | (g/L) | | Components | (mg/L) | Components | (g/L) |
| NaCl | 7.2 | | Biotin | 2.0 | Nitrilotriacetic acid | 1.5 |
| MgSO₄•7H₂O | 2.6 | | Folic acid | 2.0 | FeSO₄•7H₂O | 0.1 |
| CaCl₂•2H₂O | 1.2 | | Pyridoxine•HCl | 10.0 | MnCl₂•4H₂O | 0.1 |
| NH₄Cl | 8 | | Thiamine•HCl | 5.0 | CoCl₂•6H₂O | 0.17 |
| Yeast extract | 2.0 | | Riboflavin | 5.0 | ZnCl₂ | 0.1 |
| Vitamin solution | 10 | ml | Nicotinic acid | 5.0 | CaCl₂•6H₂O | 0.1 |
| Trace element solution | 80 | ml | Pantothenic acid | 5.0 | CuCl₂•2H₂O | 0.02 |
| Sodium bicarbonate | 2.1 | | Cyanocobalamine | 0.1 | H₃BO₃ | 0.01 |
| Na₂HPO₄ | 1.65883 | | r-aminobenzoic acid | 5.0 | Na₂MoO₄ | 0.01 |
| NaH₂PO₄ | 0.65827 | | Lipoic acid | 5.0 | Na₂SeO₃ | 0.017 |
| L-cysteine•HCl | 0.5 | | | | NiSO₄•6H₂O | 0.026 |
| 0.1% (w/v) resazurin | 200 | μl | | | NaCl | 1.0 |

Setup and Operation of Bioreactor

A bubble column reactor (BCR) was used as a basic reactor, and a continuous cell recycled reactor (CCR) was additionally applied thereto using a known method (Chang et al., 2001). Specifically, the reactor including a fermentation medium (vitamin solution, carbonate-excluding buffer) was autoclaved for 15 minutes at 121° C. After that, the reactor was sufficiently cooled under a room temperature condition, and then purged with CO₂ to remove precipitates. Finally, the headspace was purged using a CO/CO₂ mixture gas (CO:CO₂=80:20, Daedeok Gas, Korea).

Meanwhile, the gaseous substrate (CO:CO₂=4:1) of the present disclosure was sprayed through a sintered gas filter (10 μm to 16 μm pore size; Daihan Science, Korea), and a hollow fiber membrane cartridge (CFP-2-E-3MA, GE Health Care, USA) was attached to the outside of the BCR reactor to concentrate the microbial cells. Meanwhile, a medium dilution solution was used to supply nutrients during the reactor operation. Furthermore, a medium composition under an anaerobic condition was supplied through a feeding tank (glass carboy, 13 L, Pyrex, USA) to maintain a target level of medium addition rate (dilution rate), and cell-free permeate produced during medium recycling was removed at the same rate to maintain a fixed reactor volume. In addition, the pH condition inside the reactor was maintained under a condition of 6.8 to 7.2 using 2 N of NaOH.

### Analysis Method

The microbial cell concentration was analyzed through a calibration curve using optical density at 660 nm measured using a spectrophotometer (Jasco UVIDEC-610, Tokyo, Japan), and the OD₆₆₀ of “1” corresponds to 0.27 g of dry weight (CDW L⁻¹) of grown cells.

Meanwhile, fermentation products were quantified using gas chromatography (GC), and CO and CO₂ were measured using a thermal conductivity detector equipped with gas chromatography (GC). Meanwhile, the gas mixture used was supplied from Alletech.

### Experimental Results

### Example 1

A gaseous substrate including CO and CO₂ in a partial pressure ratio of 4:1 was injected to a first bioreactor including the culture medium of Table 1 and the Eubacterium limosum KIST612 strain at an initial injection rate of 0.06 vvm, and then injected so that the rate increased by 0.1 vvm per 1 g/L of an increase in the cell concentration of the strain until the rate gradually increases up to a rate of 0.86 vvm. After that, while continuously supplying highly active microorganisms having reached a cell concentration with an OD₆₆₀ value of 4 to 6 inside the first bioreactor to a second bioreactor including the culture medium of Table 2, a gaseous substrate that is the same as in the first bioreactor was injected into the second bioreactor at a rate of 0.086 vvm to 0.1 vvm and fermented, and as a result, acetate and butyrate, which are hydrocarbon products, were produced.

Meanwhile, the medium addition rate (dilution rate) in the first bioreactor was maintained at 0.12 h⁻¹, and the medium addition rate (dilution rate) in the second bioreactor was maintained at 0.017 h⁻¹.

### Example 2

After performing the process of Example 1, a sludge filtrate corresponding to 40% to 60% of the medium volume was additionally injected through the feed tank of the second bioreactor 200 hours to 300 hours after performing the product producing step in the second bioreactor.

### Example 3

After performing the process of Example 2, the partial pressure ratio of the injected CO was adjusted by replacing the gaseous substrate (including CO and CO₂ in a partial pressure ratio of 4:1) injected to the second bioreactor through a gas cylinder with a gaseous substrate including CO and CO₂ in a partial pressure ratio of 9:1 400 hours to 450 hours after performing the product producing step in the second bioreactor.

### Identification of Production Efficiency of Hydrocarbon Products (Fermentation Products)

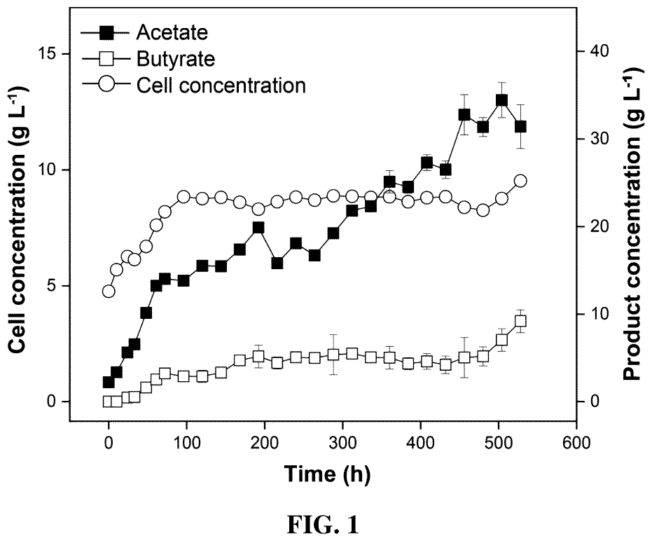

As shown in FIG. 1, it may be identified that concentrations of acetate and butyrate, which are hydrocarbon products, increase as the process according to the production method of the present disclosure progresses, and therefore, it may be identified that useful hydrocarbon products may be produced in a high yield from waste gas such as CO and CO₂ when performing the continuous process of the biomass boosting step and the product producing step of the present disclosure.

Meanwhile, referring to FIG. 1, it may be identified that the concentrations of acetate and butyrate no longer increase and remain relatively constant when 200 hours to 300 hours have passed, and it was identified that the concentration of acetate rapidly increased when additionally injecting a sludge filtrate corresponding to 40% to 60% of the medium volume injected through a feed tank of the second bioreactor when the 200 hours to 300 hours had passed.

Furthermore, referring to FIG. 1, it may be identified that, despite additional injection of the sludge filtrate when 200 hours to 300 hours have passed, the concentrations of acetate and butyrate no longer increase again and remain relatively constant when 400 hours to 450 hours have passed. The concentrations of acetate and butyrate may increase again by adjusting the partial pressure ratio of the gaseous substrate injected into the second bioreactor through a gas cylinder, and specifically, it was identified that, when a gaseous substrate including CO and CO₂ in a partial pressure ratio of 9:1 was injected into the second bioreactor when 400 hours to 450 hours had passed, the concentrations of acetate and butyrate increased again as identified in FIG. 1, and as a result, production of acetate having a maximum concentration of 34.4 g/L was identified.

### Identification of Microbial Activity Obtained by Performing Biomass Boosting Step in Separate Reactor

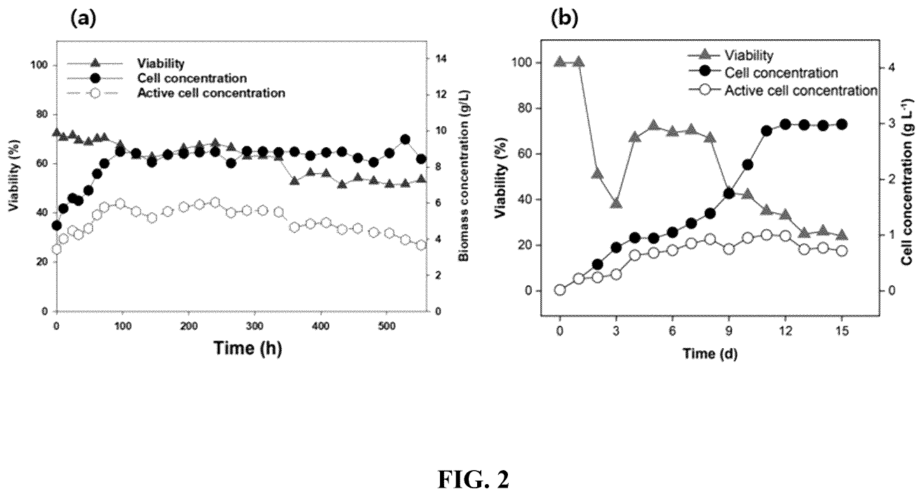

FIG. 2(a) shows results of measuring viability of the microorganisms during the process of the method for producing syngas fermentation products of the present disclosure, and referring to FIG. 2(a), it may be identified that viability of 50% to 53% is maintained throughout the process. In other words, when a biomass boosting step for microorganisms is performed in a separate reactor from a product producing step as in the present disclosure, high viability of the microorganisms may be maintained throughout the process in terms that highly active microorganisms may be continuously supplied into the second reactor in which the product producing step is actually performed, and production of acetate and butyrate, which are fermentation products, may be improved using microorganisms showing excellent activity.

On the other hand, FIG. 2(b) shows results of measuring viability of the microorganisms in a process in which a biomass boosting step and a product producing step are continuously performed in one bioreactor, unlike the present disclosure. Referring to FIG. 2(b), it may be identified that viability decreases to 40% on the third day of the process, and although the viability is recovered briefly, the activity rapidly drops as 9 days have passed after the process.

In other words, when a biomass boosting step for microorganisms and a product producing step are performed in one bioreactor, the percentage of old cells increases in the reactor as the continuous process progresses, reducing viability of the microorganisms, and as a result, production of acetate and butyrate, which are fermentation products, is reduced.

In other words, the present disclosure has an advantage in that, by performing a biomass boosting step in a separate reactor from a product producing step, highly active microorganisms may be continuously injected into a reactor in which the product producing step is performed, and production of the hydrocarbon products of the present disclosure may be maximized using the continuously supplied highly active microorganisms.

Hereinbefore, preferred embodiments of the present disclosure have been described in 10 detail, however, the scope of a right of the present disclosure is not limited thereto, and various modified and improved forms made by those skilled in the art using the basic concept of the present disclosure defined in the claims also fall within the scope of a right of the present disclosure.

What is claimed is:

1. A method for producing syngas fermentation products using highly active microorganisms, the method comprising:
a biomass boosting step of obtaining highly active microorganisms by injecting a gaseous substrate including CO into a first bioreactor;
a step of supplying the obtained highly active microorganisms to a second bioreactor; and
a product producing step of injecting a gaseous substrate into the second bioreactor, and producing hydrocarbon products by a fermenting action of the highly active microorganisms in the second bioreactor,
wherein the first bioreactor includes a first culture medium; and
the second bioreactor includes a second culture medium.

2. The method of claim 1, wherein a pH condition inside the first and second bioreactors is from 6.8 to 7.2.

3. The method of claim 1, wherein a first culture medium addition rate (dilution rate) in the first bioreactor is from 0.08 h⁻¹ to 0.15 h⁻¹.

4. The method of claim 1, wherein a second culture medium addition rate (dilution rate) in the second bioreactor is from 0.01 h⁻¹ to 0.02 h⁻¹.

5. The method of claim 1, wherein a gaseous substrate injection rate (vvm) into the first bioreactor is from 0.06 vvm to 0.86 vvm.

6. The method of claim 1, wherein a gaseous substrate injection rate (vvm) into the second bioreactor is from 0.086 vvm to 0.1 vvm.

7. The method of claim 1, wherein the first and second culture media include a compound selected from the group consisting of NaCl, MgSO₄·7H₂O, CaCl₂·2H₂O, NH₄Cl and mixtures thereof.

8. The method of claim 1, wherein the microorganism is selected from the group consisting of genus Moorella, Clostridium, Ruminococcus, Acetobacterium, Eubacterium, Butyribacterium, Oxobacter, Methanosarcina, Methanosarcina and Desulfotomaculum.

9. The method of claim 1, wherein the hydrocarbon product is selected from the group consisting of acetate, butyrate, ethanol, propanol, butanol, 2,3-butanediol, propionate, caproate, propylene, butadiene, isobutylene, ethylene and mixtures thereof.

10. The method of claim 1, wherein the highly active microorganisms have an OD₆₆₀ value in a range of 4 to 6.

11. The method of claim 1, wherein the gaseous substrate includes CO and CO₂ in a partial pressure ratio of 3.5:1 to 4.5:1.

12. A hydrocarbon product produced using the method of claim 1.

13. A system for producing syngas fermentation products using highly active microorganisms, the system comprising:
a first bioreactor;
a second bioreactor;
a gas cylinder capable of injecting a gaseous substrate including CO into the first bioreactor and the second bioreactor; and
a feed tank connected to each of the first bioreactor and the second bioreactor and capable of controlling a medium addition rate (dilution rate),
wherein highly active microorganisms obtained in the first bioreactor are supplied to the second bioreactor connected to the first bioreactor, and the gaseous substrate is injected and fermented to produce hydrocarbon products.

14. The system of claim 13, wherein, to the second bioreactor, a hollow fiber membrane is additionally connected to concentrate the microorganisms.

15. The system of claim 13, wherein the first and second bioreactors are capable of independently controlling one or more variables selected from the group consisting of the medium addition rate, the gaseous substrate injection rate, a pH inside the reactors and combinations thereof.

16. The system of claim 13, wherein a base tank is connected to the first and second bioreactors, and by the base tank, a pH inside the first and second bioreactors is maintained at 6.8 to 7.2.

17. The system of claim 13, wherein an injection rate (vvm) of the gaseous substrate injected into the first bioreactor through the gas cylinder is from 0.06 vvm to 0.86 vvm.

18. The system of claim 13, wherein an injection rate (vvm) of the gaseous substrate injected into the second bioreactor through the gas cylinder is from 0.086 vvm to 0.1 vvm.

19. The system of claim 13, wherein a first culture medium addition rate (dilution rate) in the first bioreactor adjusted through the feed tank is from 0.08 h⁻¹ to 0.15 h⁻¹.

20. The system of claim 13, wherein a second culture medium addition rate (dilution rate) in the second bioreactor adjusted through the feed tank is from 0.01 h⁻¹ to 0.02 h⁻¹.
